SEQUENCE LISTING (17 sequences)

SEQ ID NO: 1 - DNA, 22 bp, Artificial Sequence
  the nucleotide sequence of the effective sequence
aacactgattttcctggtgcta

SEQ ID NO: 2 - DNA, 12 bp, Artificial Sequence
  the nucleotide sequence of the random sequence
aaacgtgctacg

SEQ ID NO: 3 - DNA, 396 bp, Artificial Sequence
  the nucleotide sequence of the circular RNA circmiR-29b
aacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgctaaaacgtgctacgaacactgattttcctggtgcta

SEQ ID NO: 4 - DNA, 1060 bp, Artificial Sequence
  the nucleotide sequence of the 5 guided loop-forming sequence
tagaatcgccactcctgcatcatttggtctcttgagcccactatagacttaatatgtgttttggaataatcatggaaagaatgacattggcactgagaaggaacattttatggctgactctgcagccttctaggctcttccatacagctcagcaaaggcctggttatctcttcttcctgttgcacactgcctgcaacttcgaaagcaggctgtgtatacagatgtgaagaactgtgcttgaagacccatgtgaccagctgatgatctatcagaagtcccaacttgttgaaatggggggaggggaaggactgtgtcttaatgaaaacaggctttgaaagaactgcacctaaaatatgagaatactgcatcttcagtgaaataatttccttcacaaggacaaatacttttgagctttattgccaatgagcttgaagatttctctgttcccaaggaaccttggagcagtaggatttttataaaacacaataaagatgtactccaggatttaagataatcagcatgcattgtattgggattatttcagagtctgagtttcaagatgtggctcttaataaggtgattttgtcaatgaaattttttccaaagatatcatactaagtcaaatggaaaaaaaaattcacttgtgcaacaaggaagaatttacttaatgcttcaagaggtttgctttctggctgttagtatgtaccagcatgtgctcaatagaactgtgcggtaatgggacttttcattttcagggagtcatatgtgtgtcttgctccatgaacgggactacatgtagaaaatacacaaacagagagattgaattgtaaatattacaaaagattgtttagtcactgatggattcttaggataatgattccacagagattttatgatatagaaagttttggtataagtgattcaaataagttattaatgatgctttgactctagtgatgtgatttctcttcttcgtcgtctttttttttttttttaactgcaggttggagcagttggaagtcttattgtacaacatgcttcgattaagtctcccacccaa

SEQ ID NO: 5 - DNA, 1056 bp, Artificial Sequence
  the nucleotide sequence of the 3 guided loop-forming sequence
gaggtggaggggaagactttgaggacacctgtggagagctcgaattccagaacgatgaaatagtgtaagttacctggttacctgtgtgtgtgtgtgtgtgtgtgtgtgtgtgtgcctgtgtgtgtgtgtgtgtatctttttgcatgtgagtatgtactcttttaacaagtgcaattcaagaaaccacctaagaatccatcagtgactaaacaatcttttgtaatatttacaattcaatctctctgtttgtgtattttctacatgtagtcccgttcatggagcaagacacacatatgactccctgaaaatgaaaagtcccattaccgcacagttctattgagcacatgctggtacatactaacagccagaaagcaaacctcttgaagcattaagtaaattcttccttgttgcacaagtgaatttttttttccatttgacttagtatgatatctttggaaaaaatttcattgacaaaatcaccttattaagagccacatcttgaaactcagactctgaaataatcccaatacaatgcatgctgattatcttaaatcctggagtacatctttattgtgttttataaaaatcctactgctccaaggttccttgggaacagagaaatcttcaagctcattggcaataaagctcaaaagtatttgtccttgtgaaggaaattatttcactgaagatgcagtattctcatattttaggtgcagttctttcaaagcctgttttcattaagacacagtccttcccctccccccatttcaacaagttgggacttctgatagatcatcagctggtcacatgggtcttcaagcacagttcttcacatctgtatacacagcctgctttcgaagttgcaggcagtgtgcaacaggaagaagagataaccaggcctttgctgagctgtatggaagagcctagaaggctgcagagtcagccataaaatgttccttctcagtgccaatgtcattctttccatgattattccaaaacacatattaagtctatagtgggctcaagagaccaaatgatgcaggagtggcgattcta

SEQ ID NO: 6 - RNA, 23 nt, Artificial Sequence
  the nucleotide sequence of miR-29b
uagcaccauuugaaaucaguguu

SEQ ID NO: 7 - DNA, 20 bp, Artificial Sequence
  upstream primer F
gaggtggaggggaagacttt

SEQ ID NO: 8 - DNA, 20 bp, Artificial Sequence
  downstream primer R
tagaatcgccactcctgcat

SEQ ID NO: 9 - DNA, 40 bp, Artificial Sequence
  primer PGL3-R-circmiR-29b-F
cgatctaagtaagctagtgactccgtctctcatatgatcg

SEQ ID NO: 10 - DNA, 37 bp, Artificial Sequence
  primer PGL3-R-circmiR-29b-R
ccggaatgccaagctagtgtcttgcgtctctgaattc

SEQ ID NO: 11 - DNA, 60 bp, Artificial Sequence
  the inserted fragment
atgtcgacaaacgtgctaaagcttgaattcaaacgtgctacatatgtctagactcgagat

SEQ ID NO: 12 - DNA, 38 bp, Artificial Sequence
  primer Infusion-3p-F
aacgtgctacatatgtctaggaggtggaggggaagact

SEQ ID NO: 13 - DNA, 32 bp, Artificial Sequence
  Infusion-3p-R
gattcttaggtggtttcttgaattgcacttgt

SEQ ID NO: 14 - DNA, 35 bp, Artificial Sequence
  primer Infusion-5p-Inverted-F
caagaaaccacctaagaatccatcagtgactaaac

SEQ ID NO: 15 - DNA, 41 bp, Artificial Sequence
  primer Infusion-5p-Inverted-R
atgaattatctcgagtctagtagaatcgccactcctgcatc

SEQ ID NO: 16 - DNA, 26 bp, Artificial sequence
  primer Insert-5p-F
gtcgactagaatcgccactcctgcat

SEQ ID NO: 17 - DNA, 26 bp, Artificial Sequence
  primer Insert-5p-R
aagcttttgggtgggagacttaatcg

(19) United States
(12) Patent Application Publication
(10) Pub. No.: US 2022/0403384 A1
(43) Pub. Date: Dec. 22, 2022

(54) ENGINEERED CIRCULAR RNA CIRCMIR-29B AND USE THEREOF IN PREPARATION OF MEDICINE FOR TREATING MUSCLE ATROPHY

(71) Applicant: Shanghai University, Shanghai (CN)

(72) Inventors: Junjie XIAO, Shanghai (CN); Jin LI, Shanghai (CN); Yihua BEI, Shanghai (CN)

(73) Assignee: Shanghai University, Shanghai (CN)

(21) Appl. No.: 17/492,465

(22) Filed: Oct. 1, 2021

(30) Foreign Application Priority Data
Jun. 21, 2021 (CN) 202110685115.9

(51) Int. Cl. C12N 15/113; C12N 15/86; A61K 31/7088; A61P 21/00

(52) U.S. Cl. CPC C12N 15/113; C12N 15/86; A61K 31/7088; A61P 21/00; C12N 2310/532; C12N 2750/14143; C12N 2310/141; C12N 2750/14171

(57) ABSTRACT

The present disclosure belongs to the technical field of biomedicine, and provides an engineered circular RNA circmiR-29b and use thereof in preparation of a medicine for treating muscle atrophy. The present disclosure provides a circular RNA circmiR-29b including an effective sequence and a random sequence, wherein 6-13 repetitions of the effective sequence are connected in series, the random sequence is inserted between the effective sequences, and the nucleotide sequence of the effective sequence is shown in SEQ ID NO: 1. The present disclosure delivers circmiR-29b to skeletal muscle by AAV8, enabling stable expression of circmiR-29b in the skeletal muscle, thereby effectively inhibiting various types of muscle atrophy. Therefore, the present disclosure also provides a gene therapy based on the AAV8 virus vector delivering the engineered circular RNA circmiR-29b to achieve the objective of treating muscle atrophy.

### CROSS-REFERENCE TO RELATED APPLICATION

This application is a continuation of and claims priority to Chinese Patent Application No. 202110685115.9, filed 21 Jun. 2021, entitled “ENGINEERED CIRCULAR RNA CIRCMIR-29B AND USE THEREOF IN PREPARATION OF MEDICINE FOR TREATING MUSCLE ATROPHY”, the entire disclosure of which is incorporated herein by this reference.

### REFERENCE TO SEQUENCE LISTING SUBMITTED VIA EFS-WEB

The content of the ASCII text file of the sequence listing named “111203-0004-SEQUENCE-LISTING” which was created on Jan. 26, 2022, and is 6,916 bytes in size submitted electronically via EFS-Web in this U.S. non-provisional patent application is incorporated by reference in its entirety.

### TECHNICAL FIELD

The present disclosure belongs to the technical field of biomedicine, and specifically relates to an engineered circular RNA circmiR-29b and use thereof in preparation of a medicine for treating muscle atrophy.

### BACKGROUND ART

Gene therapies is the use of molecular biology methods to deliver nucleic acid fragments to specific organs, with edited genes interfering with target gene expression or directly expressing corresponding products to treat diseases. The key of gene therapies is finding a suitable target gene and choosing a suitable delivery method. Gene therapies are mainly used in gene mutation diseases, such as hemophilia, sickle cell anemia, and muscle atrophy.

Skeletal muscle controlled by human consciousness can cause myofiber contraction in the skeletal muscle through somatic nerve stimulation to enable the basic activities of the human body including flexion and extension. Skeletal muscle is an important organ of the human body's motion system Muscle atrophy represents a group of degenerative diseases of skeletal muscle cells mainly featuring myofiber loss and muscle cell protein loss. Muscle atrophy can be caused by systemic diseases such as sepsis, acquired immune deficiency syndrome (AIDS), heart failure, and kidney failure; acute physical injuries such as severe burns and large-scale trauma; cancers and other diseases.

At present, there is a gene therapy for spinal muscular atrophy (SMA) which is an autosomal negative genetic disease usually caused by mutation in the gene survival motor neuron 1 (SMN1). The gene therapy treats SMA by delivering gene fragments capable of expressing normal SMN1 protein to motor neurons, thereby enabling stable expression of SMN1 protein for a long time. However, there are many mechanisms that can cause muscle atrophy, and the gene therapy for SMA is not applicable to muscle atrophy caused by other factors. Therefore, it is necessary to find a universal gene that regulates muscle atrophy and interfere with the gene to treat muscle atrophy extensively.

In the occurrence and development of muscle atrophy, non-coding RNAs play an important role. Studies have shown that various small RNAs such as miR-1, miR-29b, miR-133, miR-23a, miR-21, miR-27, miR-628, miR-431 and miR-206 play a regulatory role in the occurrence of muscle atrophy. For example, the invention patent published as CN108690846A discloses that muscle atrophy is treated by inhibiting the gene expression of miR-29b. It also discloses that an inhibitor inhibits the expression of miR-29b in cells and tissues, or destabilizes miR-29b in cells and tissues, or reduces the activity or effective time of action of miR-29b in cells and tissues, to achieve treatment of muscle atrophy. A small interfering RNA (siRNA) can specifically bind to miR-29b, affecting the regulation of miR-29b, and thereby treating muscle atrophy. However, at present, linear siRNAs are unstable and easily degraded in cells. Moreover, they only reduce the expression of miR-29b in the body by a limited amount, which is not enough for treatment of muscle atrophy.

### SUMMARY

In view of this, an objective of the present disclosure is to provide a circular RNA circmiR-29b as a molecular sponge for miR-29b to reduce the expression of miR-29b in cells.

Another objective of the present disclosure is to provide a use of the circular RNA circmiR-29b in preparation of a medicament for preventing and/or treating muscle atrophy.

The present disclosure provides a circular RNA circmiR-29b, which includes an effective sequence and a random sequence, wherein 6-13 repetitions of the effective sequence are connected in series, the random sequence is inserted between the effective sequences;

and the nucleotide sequence of the effective sequence is shown in SEQ ID NO: 1.

In one embodiment, 10-12 repetitions of the effective sequence may be connected in series. In one embodiment, the random sequence may include 12-24 nucleotides (nt).

In one embodiment, the nucleotide sequence of the random sequence may be shown in SEQ IDNO: 2.

In one embodiment, the nucleotide sequence of the circular RNA circmiR-29b may be shown in SEQ ID NO: 3.

In one embodiment, the nucleotide sequence of the circular RNA circmiR-29b may be shown in SEQ ID NO: 3.

The present disclosure provides a recombinant adeno-associated virus (AAV) vector, wherein the circular RNA circmiR-29b is inserted in an AAV vector.

In one embodiment, the AAV vector may include an AAV8 vector.

In one embodiment, the AAV vector may further include a 5′ guided loop-forming sequence and a 3′ guided loop-forming sequence;

the nucleotide sequence of the 5′ guided loop-forming sequence may be shown in SEQ ID NO:4;

the nucleotide sequence of the 3′ guided loop-forming sequence may be shown in SEQ ID NO: 5;

and the circular RNA circmiR-29b may be inserted between the 5′ guided loop-forming sequence and the 3′ guided loop-forming sequence.

The present disclosure provides a delivery system for the circular RNA circmiR-29b, which is an AAV containing the circular RNA circmiR-29b or is obtained by package of the recombinant AAV vector.

The present disclosure provides a use of the RNA circmiR-29b, the recombinant AAV vector or the delivery system in preparation of a medicine for preventing and/or treating muscle atrophy.

The circular RNA circmiR-29b provided by the present disclosure may include an effective sequence and a random sequence, wherein 6-13 repetitions of the effective sequence are connected in series, the random sequence is inserted between the effective sequences, and the nucleotide sequence of the effective sequence is shown in SEQ ID NO: 1. The present disclosure provides an effective sequence capable of strictly complementary pairing with the nucleotide sequence of miR-29b, and an engineered circular RNA circmiR-29b. Tests show that, compared with a linear effective sequence with an miR-29b inhibitory efficiency of 44.8%, the circular RNA circmiR-29b provided by the present disclosure has an inhibitory efficiency of more than 90%, greatly reducing the expression of miR-29b and thus achieving the objective of treating muscle atrophy.

### BRIEF DESCRIPTION OF THE DRAWINGS

FIG. 1 is a schematic diagram of AAV-dMCK-circmiR-29b constructed in an embodiment of the present disclosure, in which dMCK is a muscle-specific promoter, a 5′ intron sequence is a 5′ guided loop-forming sequence, a 3′ intron sequence is a 3′ guided loop-forming sequence, an artificially synthesized circular RNA is inserted in the yellow fragment.

FIGS. 2A-2B show the results of cyclic circmiR-29b specifically reducing the miR-29b expression in an embodiment of the present disclosure, wherein FIG. 2A shows the relative miR-29b expression results in the AAV8-circmiR-29b group and the control group, and FIG. 2B shows the results of luciferase in the AAV8-circmiR-29b groups and the control groups in an embodiment of the present disclosure.

FIGS. 3A-3B show the circmiR-29b protection from muscle atrophy in terms of myotubes according to an embodiment of the present disclosure, wherein FIG. 3A shows the results of immunofluorescence staining of the AAV8-circmiR-29b group and the control group, and FIG. 3B shows the statistical graph of immunofluorescence staining results of the AAV8-circmiR-29b groups and the control groups; ***,p<0.001.

FIGS. 4A-4E show the AAV-delivered circmiR-29b protection from the muscle atrophy induced in a nerve-cutting (denervation (Den)) model in an embodiment of the present disclosure, wherein FIG. 4A shows the anatomical morphology of mouse gastrocnemius in the test of AAV8-circmiR-29b preventing muscle atrophy in the Den model, FIG. 4B shows the results of weight change of mouse gastrocnemius samples in each group, FIG. 4C shows the results of miR-29b expression in the mouse gastrocnemius in each group, FIG. 4D shows the results of the wheat germ agglutinin (WGA) staining and the statistical results of diameter of myofiber of the mouse gastrocnemius in each group, and FIG. 4E shows the results of hematoxylin and eosin (HE) staining and the statistical results of diameter of myofiber of the mouse gastrocnemius in each group; *,p<0.0S, **,p<0.01, ***,p<0.001.

FIGS. 5A-5E show the AAV-delivered circmiR-29b protection from the muscle atrophy induced in an immobilization (IMO) model, wherein FIG. 5A shows the anatomical morphology of mouse gastrocnemius in the test of AAV8-circmiR-29b preventing muscle atrophy in the IMO model, FIG. 5B shows the results of weight change of the mouse gastrocnemius samples in each group, FIG. 5C shows the results of miR-29b expression in the mouse gastrocnemius in each group, FIG. 5D shows the results of the WGA staining and the statistical results of diameter of myofiber of the mouse gastrocnemius in each group, and FIG. 5E shows the results of HE staining and the statistical results of diameter of myofiber of the mouse gastrocnemius in each group; *,p<0.0S, **, p<0.01, ***,p<0.001.

FIGS. 6A-6F show the delivered circmiR-29b protecting from the muscle atrophy induced in a angiotensin II (Angil) sustained release pump model (Angil model), wherein FIG. 6A shows the anatomical morphology of mouse gastrocnemius in the test of AAV8-circmiR-29b preventing muscle atrophy in the Angil model, FIG. 6B shows the grip test results at the end of the test in each mouse model group, FIG. 6C shows the results of weight change of the mouse gastrocnemius samples in each group, FIG. 6D shows the results of miR-29b expression in the mouse gastrocnemius in each group, FIG. 6E shows the results of the WGA staining and the statistical results of diameter of myofiber of the mouse gastrocnemius in each group, and FIG. 6F shows the results of HE staining and the statistical results of diameter of myofiber of the mouse gastrocnemius in each group; *,p<0.0S, **,p<0.01, ***,p<0.001.

FIGS. 7A-7F show the delivered circmiR-29b treating the muscle atrophy induced in a spiral wire immobilization (SWI) model, wherein FIG. 7A shows the gastrocnemius atrophy in mice on different days since construction of the SWI model, FIG. 7B shows the anatomical morphology of mouse gastrocnemius in the test of AAV8-circmiR-29b treating muscle atrophy caused in the SWI model, FIG. 7C shows the results of weight change of the mouse gastrocnemius samples in each group, FIG. 7D shows the results of miR-29b expression in the mouse gastrocnemius in each group, FIG. 7E shows the results of the WGA staining and the statistical results of diameter of myofiber of the mouse gastrocnemius in each group, and FIG. 7F shows the results of HE staining and the statistical results of diameter of myofiber of the mouse gastrocnemius in each group; *,p<0.0S, **, p<0.01, ***,p<0.001.

FIG. 8 shows a standard curve for detecting of viral titer in an embodiment of the present disclosure.

### DETAILED DESCRIPTION OF THE EMBODIMENTS

The present disclosure provides a circular RNA circmiR-29b including an effective sequence and a random sequence, wherein 6-13 repetitions of the effective sequence are connected in series, the random sequence is inserted between the effective sequences, and the nucleotide sequence of the effective sequence is shown in SEQ ID NO: 1.

In the present disclosure, the effective sequence may be a sequence strictly complementary pairing with miR-29b. The effective sequences may be connected in 6-13 repetitions in series to increase a chance of binding miR-29b. In the present disclosure, the repetitions of the effective sequence may be preferably 10-12 in number. The test of the present disclosure proves that the miR-29b expression can be effectively reduced by increasing the repetition number of the effective sequence, thereby improving the inhibition efficiency to enable better treatment of muscle atrophy, and a larger number of repetition results in a better prevention or treatment effect.

In the present disclosure, the random sequence may preferably include 12-24 nt. tests prove that the random sequence including 12-24 nt has the best effect, and as a spacer sequence is further extended (more than 24 nt), the inhibitory effect thereof is significantly weakened. The present disclosure has no special requirements for the specific nucleotide sequence of the random sequence, and a random sequence well-known in the art is sufficient. In an embodiment of the present disclosure, the nucleotide sequence of the random sequence may be preferably shown in SEQ ID NO: 2. In an embodiment of the present disclosure, an effective sequence used in 12 repetitions is taken as an example to illustrate the effect of the circular RNA The nucleotide sequence of the circular RNA circmiR-29b may be preferably shown in SEQ ID NO: 3.

The present disclosure provides a recombinant AAV vector, wherein the circular RNA circmiR-29b is inserted in an AAV vector.

In the present disclosure, the AAV vector may preferably include an AAV8 vector. Since AAV8 can accurately target muscle cells and deliver circular RNA circmiR-29b gene fragments to the muscle cells, the AAV8 vector is selected to construct the recombinant AAV vector. In one embodiment, the AAV vector may further include a 5′ guided loop-forming sequence and a 3′ guided loop-forming sequence, both of which may be derived from exon 2 of the Slc8al gene of a mouse genome. The nucleotide sequence of the 5′ guided loop-forming sequence may be shown in SEQ ID NO: 4, and the nucleotide sequence of the 3′ guided loop-forming sequence may be shown in SEQ ID NO: 5. The circular RNA circmiR-29b may be inserted preferably between the 5′ guided loop-forming sequence and the 3′ guided loop-forming sequence. In an embodiment of the present disclosure, the recombinant AAV8 vector is taken as an example to illustrate the method for delivering the circular RNA circmiR-29b by the delivery system A map of the recombinant AAV8 vector may be shown in FIG. 1, which contains a dMCK promoter enabling efficient expression of the circular RNA circmiR-29b.

In the present disclosure, a method for constructing a recombinant vector containing the circular RNA circmiR-29b may preferably include the following steps:

step 1): inserting a fragment (SEQ ID NO: 10) between EcoRI restriction sites on an AAV-dMCK-EGFP vector, linearizing the vector with XbaI, inserting a 3′ guided loop-forming sequence (186 nt) and a reverse 5′ guided loop-forming sequence (870 nt) by homologous recombination to obtain a recombinant plasmid containing guided loop-forming sequences, and linearizing the recombinant plasmid containing guided loop-forming sequences through EcoRI single enzyme digestion;

step 2): artificially synthesizing the RNA circmiR-29b and cloning it into a T plasmid to obtain a recombinant T plasmid containing the RNA circmiR-29b;

step 3): amplifying a 5′ guided loop-forming sequence (1060 nt) with a polymerase chain reaction (PCR) method, digesting a PCR product with EcoRI, ligating a linearized T vector and the PCR product through a reaction with a T4 ligase at room temperature for 30 min to obtain a recombinant T plasmid, linearizing the recombinant T plasmid through digestion with EcoRI and NdeI, and ligating a circmiR-29b fragment and a linearized recombinant plasmid containing guided loop-forming sequences with a T4 ligase to obtain a recombinant vector containing the circular RNA circmiR-29b (AAV-dMCK-circmiR-29b plasmid).

In the present disclosure, the AAV-dMCK-EGFP plasmid has been reported in the prior art, see Prevention of Muscle Wasting by CRISPR/Cas9-mediated Disruption of Myostatin In J/ivo; doi: 10.1038/Mt.2016.192.; CRISPR/Cas9-Mediated miR-29b Editing as a Treatment of Different Types of Muscle Atrophy in Mice; doi: 10.1016/j.ymthe.2020.03.005.

In the present disclosure, the inserted fragment 5′-ATGTCGACAAACGTGCTAAAGCTTGAATTCAAACGTGCTACATATGTCTAGACTCGAG AT-3′ (SEQ ID NO: 11) can add multiple cloning sites, and added restriction endonuclease digestion sites are Sall, Accl, HinclI, HindIII, EcoRI, NdeI, XbaI, and Xholl in sequence.

Primers for amplifying the 3′ guided loop-forming sequence (186 nt) may be as follows:

Infusion-3p-F:
(SEQ ID NO: 12)
AACGTGCTACATATGTCTAGGAGGTGGAGGGGAAGACT;
Infusion-3p-R:
(SEQ ID NO: 13)
GATTCTTAGGTGGTTTCTTGAATTGCACTTGT.

Primers for amplifying the reverse 5′ guided loop-forming sequence (870 nt) may be as follows:

Infusion-5p-Inverted-F:
(SEQ ID NO: 14)
CAAGAAACCACCTAAGAATCCATCAGTGACTAAAC;
Infusion-5p-Inverted-R:
(SEQ ID NO: 15)
ATGAATTATCTCGAGTCTAGTAGAATCGCCACTCCTGCATC.

Primers for amplifying the 5′ guided loop-forming sequence (1060 nt) with a PCR method may be as follows:

Insert-5p-F:
(SEQ ID NO: 16)
GTCGACTAGAATCGCCACTCCTGCAT
Insert-5p-R:
(SEQ ID NO: 17)
AAGCTTTTGGGTGGGAGACTTAATCG

In the present disclosure, the above PCR amplification systems are all 50 μL reaction systems with specific components as follows:

| | |
|---|---|
| KOD-neo-plus | 1 μL |
| Primer mix F + R (5 nM) | 3 μL |
| Mg2SO4 | 3 μL |
| 1O × Buffer | 5 μL |
| dNTPs | 5 μL |
| gDNA | 4 μL |
| Double distilled water | 29 μL |

A PCR amplification procedure may be shown in Table 1 below:

| | | | | |
|---|---|---|---|---|
| 95° | c. | 5 | min | 2re-denaturation |
| 95° | c. | 10 | s | reaction cycle |
| 60° | C. | 10 | s | (35 cycles) |
| 68° | C. | 30 | s | |
| n° | c. | 1O | min | sufficient |
| 4° | c. | 5 | min | amplification |

The present disclosure provides a delivery system of the circular RNA circmiR-29b, which is an AAV containing the circular RNA circmiR-29b or is obtained by packaging the recombinant AAV vector.

The present disclosure has no specific limitation on a method for packaging the AAV vector containing the circular RNA circmiR-29b, and a scheme for packaging an AAV well known in the art can be employed.

The present disclosure provides a use of the RNA circmiR-29b, the recombinant AAV vector or the delivery system in preparation of a medicine for preventing and/or treating muscle atrophy.

In the present disclosure, a dosage form of the medicine may include preferably an injectable formulation. The injectable formulation may include the delivery system. The delivery system may have a concentration of preferably 1×1011 VG/mouse-Ix 1012 VG/mouse, more preferably 1×1012 VG/mouse. The injectable formulation may be dosed 1×1012 VG/mouse for one time, with effects seen 3 weeks later.

In the present disclosure, types of the muscle atrophy include muscle atrophy caused by Den model, muscle atrophy caused by disuse (IMO model, SWI model), and muscle atrophy caused by heart failure (Angil model).

In the present disclosure, the circmiR-29b is transcribed and circularized by a eukaryotic cell itself, where a sequence guiding its correct circularization may be selected from the natural circular RNA Slc8al circRNA, and the DNA fragment may be delivered to a muscle tissue by the AAV8 vector. Therefore, the present disclosure also provides a use of circmiR-29b in prevention and/or treatment of muscle atrophy.

The engineered circular RNA circmiR-29b and use thereof in preparation of a medicine for treating muscle atrophy provided by the present disclosure will be described in detail below in combination with embodiments, but these embodiments should not be construed as limiting the scope of the present disclosure.

### EXAMPLE 1

Method for constructing a recombinant AAV vector

1. Design of circular RNA circmiR-29b fragment

According to the nucleotide sequence of miR-29b (UAGCACCAUUUGAAAUCAGUGUU, SEQ ID NO: 6), an effective sequence of miR-29b (AACACTGATTTTCCTGGTGCTA, SEQ ID NO: 1) was designed. Repetition was carried out for 12 times in a row, with 11 random sequences (AAACGTGCTACG, SEQ ID NO: 2) inserted between the repetitions to obtain a sequence of an engineered circular RNA

(AACACTGATTTTCCTGGTGCTAAAACGTGCTACGAACACTGATTTTCCT
GGTGCTAAAACGTGCTACGAACACTGATTTTCCTGGTGCTAAAACGTGCT
ACGAACACTGATTTTCCTGGTGCTAAAACGTGCTACGAACACTGATTTTC
CTGGTGCTAAAACGTGCTACGAACACTGATTTTCCTGGTGCTAAAACGTG
CTACGAACACTGATTTTCCTGGTGCTAAAACGTGCTACGAACACTGATTT
TCCTGGTGCTAAAACGTGCTACGAACACTGATTTTCCTGGTGCTAAAACG
TGCTACGAACACTGATTTTCCTGGTGCTAAAACGTGCTACGAACACTGAT
TTTCCTGGTGCTAAAACGTGCTACGAACACTGATTTTCCTGGTGCTA,
SEQ ID NO: 3).

2. Method for constructing a recombinant vector

The above engineered circular sequence, the circmiR-29b sequence, was added with EcoRI and NdeI restriction sites at both ends. The fragment was artificially synthesized by Beijing Genomics Institution (BGI) and cloned into a T vector to obtain a T vector containing the circmiR-29b sequence.

An AAV-dMCK-circmiR-29b plasmid was constructed as follows:

The AAV-dMCK-EGFP (this plasmid was given by the research group of Ding Qiurong, Institute of Nutrition, Chinese Academy of Sciences) was used as a plasmid construction backbone. The sequence 5′-atgtcgacaaacgtgctaaagcttgaattcaaacgtgctacatatgtctagactcgagat-3′ (SEQ ID NO: 11) was inserted between the EcoRI restriction sites to add multiple cloning sites. Added restriction endonuclease digestion sites were Sall, Accl, HinclI, Hindlll, EcoRI, NdeI, XbaI, and Xholl in sequence.

Then, the vector was linearized with Xbal. The two fragments, the 3′ intron sequence (186 nt) and the 5′ inverted intron sequence (870 nt), were amplified by PCR with designed primers and cloned into the vector by homologous recombination. The pnmers used for PCR were:

Infusion-3p-F:
(SEQ ID NO: 12)
AACGTGCTACATATGTCTAGGAGGTGGAGGGGAAGACT;
Infusion-3p-R:
(SEQ ID NO: 13)
GATTCTTAGGTGGTTTCTTGAATTGCACTTGT;
Infusion-5p-Inverted-F:
(SEQ ID NO: 14)
CAAGAAACCACCTAAGAATCCATCAGTGACTAAAC;
and Infusion-5p-Inverted-R:
(SEQ ID NO: 15)
ATGAATTATCTCGAGTCTAGTAGAATCGCCACT CCTGCATC.

Then, the plasmid was linearized through EcoRI single enzyme digestion, and the 5′ intron sequence (1060 nt) was amplified with designed primers. Primers for amplifying the 5′ guided loop-forming sequence (1060 nt) were as follows:

Insert-5p-F:
(SEQ ID NO: 16)
GTCGACTAGAATCGCCACTCCTGCAT;
Insert-5p-R:
(SEQ ID NO: 17)
AAGCTTTTGGGTGGGAGACTTAATCG.

The above PCR amplification systems were all 50 μL reaction systems with specific components as follows:

| | |
|---|---|
| KOD-neo-plus | 1 μL |
| Primer mix (F + R, 5 nM) | 3 μL |
| Mg2SO4 | 3 μL |
| 1O × Buffer | 5 μL |
| dNTPs | 5 μL |
| gDNA | 4 μL |
| Double distilled water | 29 μL |

A PCR amplification procedure was shown in Table 1:

| | | | | |
|---|---|---|---|---|
| 95° | c. | 5 | min | 12re-denaturation |
| 95° | c. | 10 | s | reaction cycle |
| 60° | C. | 10 | s | (35 cycles) |
| 68° | C. | 30 | s | |
| n° | c. | 1O | min | sufficient |
| 4° | c. | 5 | min | amplification |

After a PCR product was digested by EcoRI, the linearized plasmid and the PCR product were ligated through a reaction with T4 ligase at room temperature for 30 min. Finally, the plasmid was linearized with EcoRI and Ndel. A circmiR-29b fragment synthesized by BGI was digested from the T vector with EcoRI and NdeI, separated and purified by gel electrophoresis, and then ligated with the linearized plasmid through a reaction with T4 ligase at room temperature for 30 mm.

### EXAMPLE 2

A method for packaging AAV8-dMCK-circmiR-29b virus included specific steps as follows:

293T cells were inoculated in a dulbecco's modified eagle medium (DMEM) complete medium containing 10% fetal bovine serum (FBS) and 1% penicillin-streptomycin solution for culture. The 293T cells were seeded in a 10 cm cell culture dish at a density of 4 million cells per culture dish. 24 h later, 1 mL of serum-free DMEM medium containing 10 μg AAV8, 10 μg pAAV-dMCK-circmiR-29b, 10 μg Helper, and 90 μg PEI MAX was added to each petri dish for transfection. 12 h after the transfection, the DMEM complete medium was replaced with a fresh one, and the viruses in the cells and cultures was collected 48 h later. Collection of virus in culture medium: 25 mL of 40% PEG-8000 was added to every 100 mL medium, stirred overnight at 4° C., and centrifuged at 2,800 g at 15° C. for 15 min. 1 mL of cell lysis buffer was added to the virus pellet for resuspension. Collection of virus in cells: the cell pellet was resuspended in 5 mL of cell lysis buffer and placed in a refrigerator at −80° C. and a water bath at 37° C. alternatively to thaw the cells 3 times. The virus suspension in the culture medium was mixed with the thawed cell suspension, added with 1 M MgCh to a final concentration of 1 mM and Benzonase to a final concentration of 250 U/mL, incubated at 37° C. for 45 min and centrifuged at 4° C. and 4,000 rpm for 4 min. A supernatant was then taken. The virus was purified through iodixanol gradient density centrifugation. A schematic diagram of the purified virus AAV8 virus carrying a target gene was shown in FIG. I.

Method for detecting virus titer: the viral vector-based plasmid pAAV-dMCK-circmiR-29b was diluted to 1 ng/μL. The plasmid was diluted in a gradient of 2 folds for 13 times to obtain concentrations of the standard samples 1-14. A standard curve was created with 2-fold standard DNA dilution. 5 μL of the purified virus AAV8-dMCK-circmiR-29b prepared above was taken and treated with a kit according to the instructions as follows: viral gDNA was extracted, diluted to 50 μL of viral gDNA, and further diluted by 100 folds. Then, the viral titer was detected with the qPCR method.

The qPCR reaction system was as follows:

| | | |
|---|---|---|
| SYBRGreen | 5 | μL |
| Upstream primer F and downstream | 0.5 | μL |
| primer R mixture (10 μM) | | |
| ddH20 | 2.5 | μL |
| Standard DNA diluted solution or | 2 | μL |
| viral genomic DNA | | |
| Total volume | 10 | μL |

The sequences of upstream primer F and downstream primer R for use in the qPCR were as follows:

upstream primer F:
(SEQ ID NO: 7)
GAGGTGGAGGGGAAGACTTT;
downstream primer R:
(SEQ ID NO: 8)
TAGAATCGCCACTCCTGCAT.

A qPCR procedure was as follows: 40 cycles of pre-denaturation at 95° C. for 10 min, denaturation at 95° C. for 15 s, and annealing at 60° C. for 30 S.

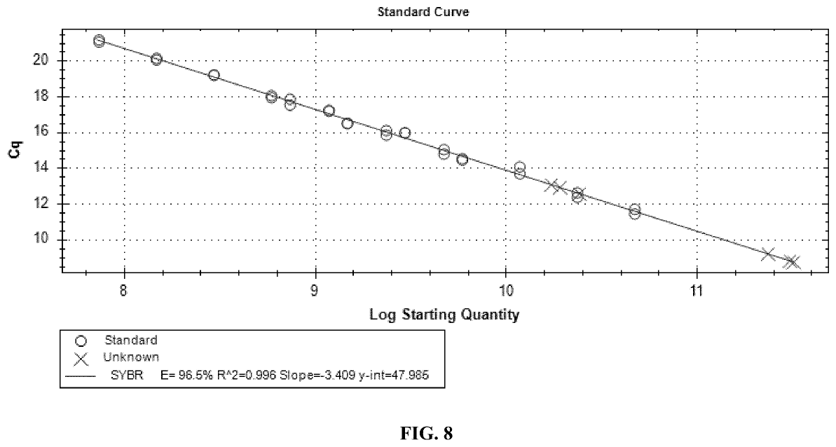

Since there was a linear relationship between the number of cycles and the logarithm of the concentration of standard DNA dilution (see FIG. 8), the viral titer was obtained by linear fitting. The viral titer was 3.057×1013 VG/mL.

After detecting titer, the AAVS-circmiR-29 virus can be directly used in animal tests or frozen at −80° C.

### EXAMPLE 3

1. A method for culturing C2C12 cells to be differentiated into myotubes included the following specific steps: C2C12 cells were inoculated in a DMEM complete medium containing 10% FBS and 1% penicillin-streptomycin solution. A medium for inducing differentiation of C2C12 cells was DMEM medium containing 2% horse serum and 1% penicillin-streptomycin solution. The medium was replaced every 24 h during differentiation, and the differentiation generally completed in 3-5 d. The cells were cultured in a thermostat incubator containing 5% CO2 at 37° C. with a saturated humidity.

2. Verification of targeting and functioning properties of AAVS-circmiR-29 virus m delivering circmiR-29b: the AAVS-circmiR-29 virus was used to deliver the target gene fragment capable of expressing circmiR-29b into muscle cells, and then the fluorescence quantitative PCR method was adopted to detect the miR-29b expression.

A specific method was as follows: TRizol Reagent was used to extract total RNA in cells. 400 ng RNA was taken and added with a specific reverse transcription (RT) primer (purchased from Ruibo). An RT kit from Bio-rad was used to carry out the RT test with the RT reaction system as follows:

| | |
|---|---|
| 400 ng RNA | 5.5 μL |
| miR-29b RT primer (5 nM) | 1 μL |
| 5s RT primer (5 nM) | 1 μL |

The miR-29b RT primer was purchased from Ruibo.

The volume of the mixture was 7.5 μL. Then the stem-loop structure of the RT primer was fully opened at 70° C. for 10 min, and annealing was carried out at 4° C. for 5 min to ensure sufficient binding between the miRNA and the specific RT primer.

2 μL 5xiScript reaction mixture and 0.5 μL iScript reverse transcriptase were added to carry out the following procedure:

| | | |
|---|---|---|
| Priming | 25° C. | 5 min |
| RT | 46° C. | 20 min |
| RT inactivation | 95° C. | 1 min |

RT product was diluted 400 times and detected for miR-29b expression by qPCR with a specific method as follows.

A 10 μL qPCR reaction system was prepared:

| | |
|---|---|
| SYBR Green | 5 μL |
| Primer mix (F + R, 5 nM) | 1 μL |
| cDNA (diluted) | 4 μL, |

wherein the upstream and downstream primers of miR-29b and internal reference 5s were purchased from Ruibo.

A qPCR procedure was as follows:

| | | | | |
|---|---|---|---|---|
| 95° | c. | 5 | min | pre-denaturation |
| 95° | c. | 10 | s | reaction cycle |
| 60° | C. | 15 | s | (40 cycles) |
| n° | c. | 10 | s | |
| 95° | c. | 15 | s | dissolving |
| 60° | C. | 60 | s | curve |
| 95° | c. | 15 | s | |

3. Detecting of specificity of circmiR-29b to miR-29b by luciferase binding assay

A test method with the luciferase reporter gene was as follows: 293T cells were seeded into 12 wells at a concentration of 100000 cells/mL. When the cell density reached about 80%, a constructed PGL-circmiR-29b plasmid and a Renilla plasmid were added in 1 μg/well and transfected into the 293T cells. After 12 h, a medium was replaced, and the cells were transfected with miR-29a, miR-29b, or miR-29c mimics respectively with a final concentration of 50 nM. The medium was replaced 6-8 h after transfection. 24 h later, the cells were collected and detected for specific binding of miR-29 family to circmiR-29b with Promega's Dual-Glo® dual luciferase reporter gene detection kit.

A method for constructing the PGL-circmiR-29b plasmid was as follows: a circmiR-29b fragment was cloned into PGL3-basic by linearizing the PGL3-basic plasmid with XbaI restriction enzyme, amplifying the circmiR-29b fragment by PCR, and cloning the circmiR-29b fragment into the linearized vector by homology recombination.

PCR pnmers were:

PGL3-R-circmiR-29b-F
(SEQ ID NO: 9)
CGATCTAAGTAAGCTAGTGACTCCGTCTCTCATATGATCG;
PGL3-R-circmiR-29b-R:
(SEQ ID NO: 10)
CCGGAATGCCAAGCTAGTGTCTTGCGTCTCTGAATTC.

A PCR amplification system was a 50 μL reaction system with specific components as follows:

| | |
|---|---|
| KOD-neo-plus | 1 μL |
| Primer mix (F + R, 5 nM) | 3 μL |
| Mg2SO4 | 3 μL |
| 1O × Buffer | 5 μL |
| dNTPs | 5 μL |
| circmiR-29b plasmid template | 1 μL |
| (synthesized by BGI) | |
| Double distilled water | 32 μL |

A PCR amplification procedure was shown in Table 1 below:

| | | | | |
|---|---|---|---|---|
| 95° | c. | 5 | min | pre-denaturation |
| 95° | c. | 10 | s | reaction cycle |
| 60° | C. | 10 | s | (35 cycles) |
| 68° | C. | 30 | s | |
| n° | c. | 1O | min | sufficient |
| 4° | c. | 5 | min | amplification |

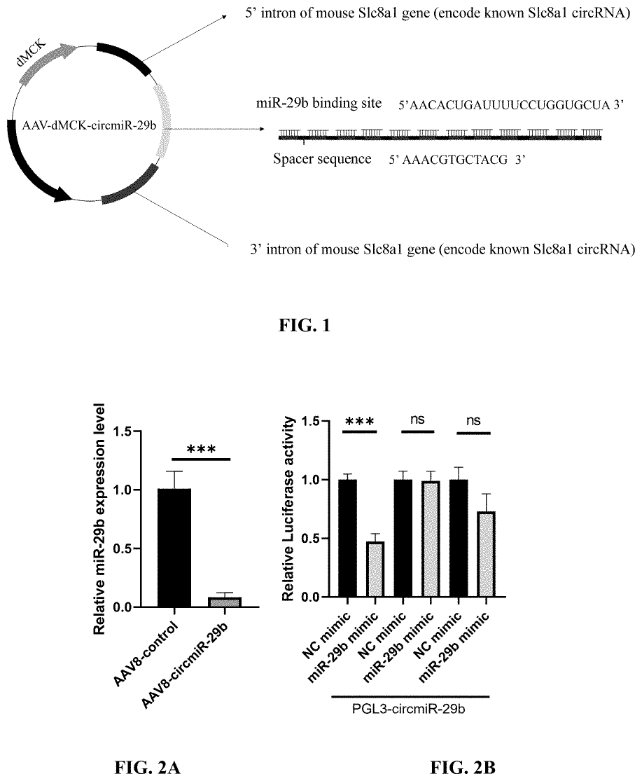

The results show in FIG. 2. Results in FIG. 2 show that circmiR can specifically reduce the miR-29b expression without affecting other members in the miR-29 family. The circmiR-29b delivered by AAV have an inhibitory effect on miR-29b. The AAV virus is selected to infect C2C12 differentiated myotubes at multiplicity of infection (MOI)=106, and the miR-29b expression is detected 48 h after virus infection. The circmiR-29b significantly reduces the miR-29b expression (FIG. 2A). The results of luciferase assay show that circmiR-29b specifically adsorb miR-29b without affecting miR-29a and miR-29c in the same family (FIG. 2B).

### EXAMPLE 4

1. C2C12 differentiated myotubes were transfected by AAV8-circmiR-29 virus. A gene fragment capable of synthesizing circmiR-29b was integrated into an AAV8 vector and packaged into an AAV8 virus with the help of AAV8 capsid plasmid and Helper plasmid. After separation and purification, the AAV viral titer was detected. The virus was added to the cell culture medium with MOI=106. 48 h later, dexamethasone (Dex, 50 μM) was added. Then, 24 h later, the diameter of myotube was detected by immunofluorescence staining.

2. Construction of Dex induced muscle atrophy model at cellular level

The myotubes differentiated from C2C12 cells were grouped as follows: group 1 was a control virus+PBS group; group 2 was a control virus+Dex group; group 3 was an AAV8-circmiR-29b virus+PBS group; group 4 was an AAV8-circmiR-29b virus+Dex group. The AAV8 virus containing circmiR-29b (or a control virus, that is, a virus without circmiR-29b) was added to the cell culture medium to infect the myotubes with MOI=106. The differentiation medium was replaced with a fresh one after 12 h. After culturing in the incubator for 36 h, the myotubes were added with Dex (50 μM) or PBS. 24 h later, the medium was removed, and the cells were fixed with paraformaldehyde and subjected to immunofluorescence staining with MF-20 antibody to detect the diameter of myotube.

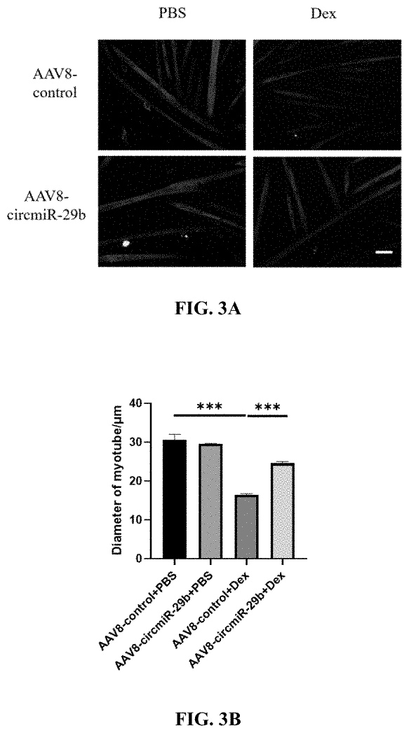

Results are shown in FIG. 3. It is detected by immunofluorescence staining that circmiR-29b enabled protection from muscle atrophy and reversed the decrease in myotube diameter caused by Dex (50 μM) (FIG. 3A and FIG. 3B). AAV delivered circmiR-29b enabled protection from muscle atrophy in terms of myotube.

### EXAMPLE 5

1. The mouse gastrocnemius was injected with AAVS-circmiR-29 virus. A gene fragment capable of synthesizing circmiR-29b was integrated into an AAV8 vector and packaged into an AAV8 virus with the help of AAV8 capsid plasmid and Helper plasmid. After separation and purification, the AAV viral titer was determined. The virus was injected into the gastrocnemius in situ at a dose of 1×1012 VG/mouse. 3 weeks later, construction of muscle atrophy model was carried out.

2. Construction of Den induced muscle atrophy model.

Wild-type male mice of 8-10 weeks old were injected intraperitoneally with 1% sodium pentobarbital at a dose of 10 μL/g for anesthesia. After anesthesia, the mice were fixed on a thermostat blanket with their abdomen facing down using a medical tape, and the hairs on the root of thigh of the right hind limb were removed with depilatory cream. The depilated part was sterilized with 75% ethanol, and then the epidermis was cut to expose the muscle tissue with microscissors. A blunt separation method was used to separate the deep muscles to expose the sciatic nerve. When the nerve was found, curved tweezers were used to pick up the sciatic nerve and microscissors were used to cut a segment of nerve 3-5 mm long to ensure that there was no possibility for nerve reconnection. Subsequently, the muscle and epidermis were sutured separately, and the mice were kept warm on the thermostat blanket before resuscitation. Then the mice were transferred to cages and raised for 7 d before they were sacrificed. The mice were measured for body weight, tibia length, and gastrocnemius weight, and then the gastrocnemius samples were kept for subsequent detection.

A specific method was as follows:

The test included 4 groups: the control virus+sham operation group, the control virus+Den group, the AAV8-circmiR-29b virus+sham operation group, and the AAV8-circmiR-29b virus+Den group.

3 weeks before the Den model was constructed, the mice were treated by intramuscular injection with a disposable 1 mL sterile syringe with a specific method as follows:

for group 1, the control virus+sham operation group, the mouse gastrocnemius was injected with the control virus at a dose of 50 μL/mouse, and the sham operation was performed 3 weeks later;

for group 2, the control virus+Den group, the mouse gastrocnemius was injected with the control virus at a dose of 50 μL/mouse, and the Den surgery was performed 3 weeks later;

for group 3, the AAV8-circmiR-29b virus+sham operation group, the mouse gastrocnemius was injected with the AAV8-circmiR-29b virus, and the sham operation was performed 3 weeks later; and

for group 4, the AAV8-circmiR-29b virus+Den group, the mouse gastrocnemius was injected with the AAV8-circmiR-29b virus, and the Den surgery was performed 3 weeks later.

1 week later, the test was over, and the mice were sacrificed. The gastrocnemius was obtained through dissection and weighed with an analytical balance. Then the muscle samples were subjected to WGA staining and HE staining to calculate the change in the cross-sectional area of myofiber. Meanwhile, tissue samples were taken. Total RNA of gastrocnemius tissue was extracted, and the change of miR-29b expression was detected by fluorescence quantitative PCR.

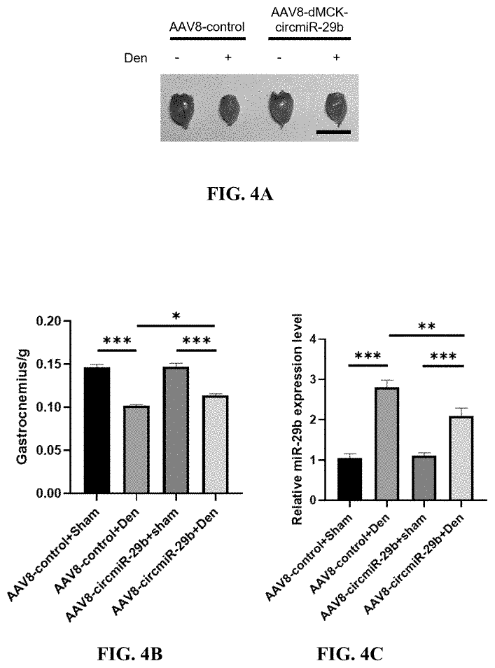

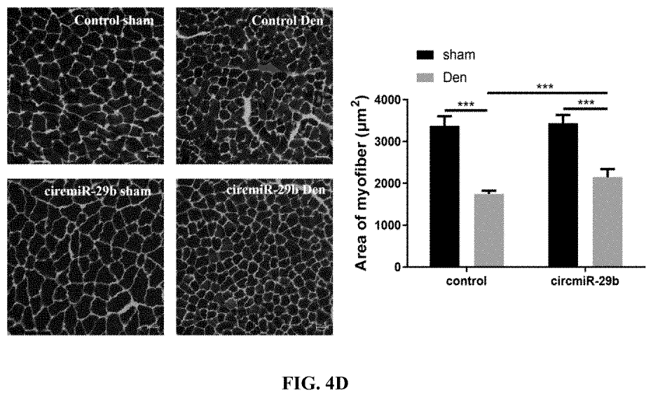

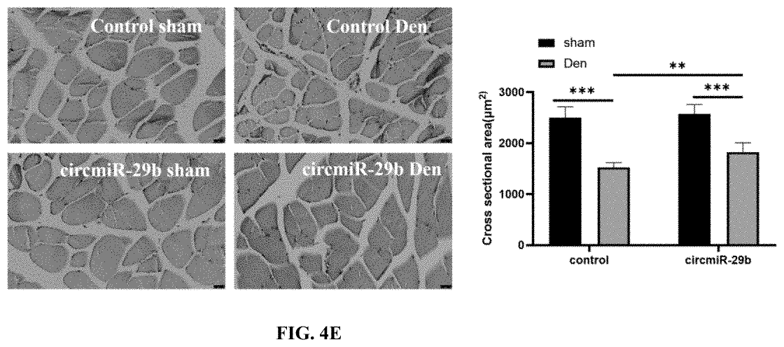

The results are shown in FIG. 4. The mouse gastrocnemius samples are significantly atrophied in the Den model, and circmiR-29b can significantly protect the muscle from weight loss (FIG. 4A and FIG. 4B). Fluorescence quantitative PCR results show that after the Den model mice are injected with AAV8-circmiR-29b, the miR-29b expression is significantly reduced (FIG. 4C). WGA and HE staining show that after the Den model mice are injected with AAV8-circmiR-29b, the diameter of myofiber is obviously improved (FIG. 4D and FIG. 4E). The circmiR-29b delivered by AAV enable protection from the muscle atrophy induced by the nerve-cutting (Den) model.

### EXAMPLE 6

1. The mouse gastrocnemius was injected with AAVS-circmiR-29 virus. A gene fragment capable of synthesizing circmiR-29b was integrated into an AAV8 vector and packaged into sufficient AAV8 viruses with the help of AAV8 capsid plasmid and Helper plasmid. After separation and purification, the AAV viral titer was determined. The virus was injected into the gastrocnemius in situ at a dose of 1×1012 VG/mouse. 3 weeks later, construction of muscle atrophy model was carried out.

2. Construction of IMO induced muscle atrophy model.

Wild-type male mice of 8-10 weeks old were injected intraperitoneally with 1% sodium pentobarbital at a dose of 10 μL/g for anesthesia. After anesthesia, the mice were fixed on a thermostat blanket with their abdomen facing down using a medical tape, and the hairs on the root of thigh of the right hind limb were removed with depilatory cream. 75% ethanol was used to disinfect the depilated part, and then an orthopedic fixation needle was inserted from the achilles tendon of the right hind limb and moved up to the tibia of the mouse, so that the right hind limb of the mouse could not be flexed and extended. The excess portion of the orthopedic fixation needle was cut off, and the inserted portion of the needle was sterilized. The mice were kept warm on the thermostat blanket before resuscitation. Then the mice were transferred to cages and raised for 7 d before they were sacrificed. The mice were measured for body weight, tibia length, and gastrocnemius weight, and then the gastrocnemius samples were kept for subsequent detection. 1 week after the model was constructed, the mice were dissected, and the gastrocnemius was weighed. The weight of the gastrocnemius with successfully constructed model decreased significantly.

A specific method was as follows:

3 weeks before the IMO model was constructed, the mice were treated by intramuscular injection with a disposable 1 mL sterile syringe. The test included 4 groups, and was carried out with a specific method as follows:

for group 1, the control virus+sham operation group, the mouse gastrocnemius was injected with the control virus at a dose of 50 μL/mouse, and the sham operation was performed 3 weeks later;

for group 2, the control virus+IMO group, the mouse gastrocnemius was injected with the control virus at a dose of 50 μL/mouse, and the IMO surgery was performed 3 weeks later;

for group 3, the AAV8-circmiR-29b virus+sham operation group, the mouse gastrocnemius was injected with the AAV8-circmiR-29b virus, and the sham operation performed 3 weeks later; and

for group 4, the AAV8-circmiR-29b virus+IMO group, the mouse gastrocnemius was injected with the AAV8-circmiR-29b virus, and the IMO surgery was performed 3 weeks later.

1 week later, the test was over, and the mice were sacrificed. The gastrocnemius was obtained through dissection and weighed with an analytical balance. Then the muscle samples were subjected to WGA staining and HE staining to calculate the change of the cross-sectional area of myofiber. Meanwhile, tissue samples were taken. Total RNA of gastrocnemius tissue was extracted, and the change in miR-29b expression was detected by fluorescence quantitative PCR.

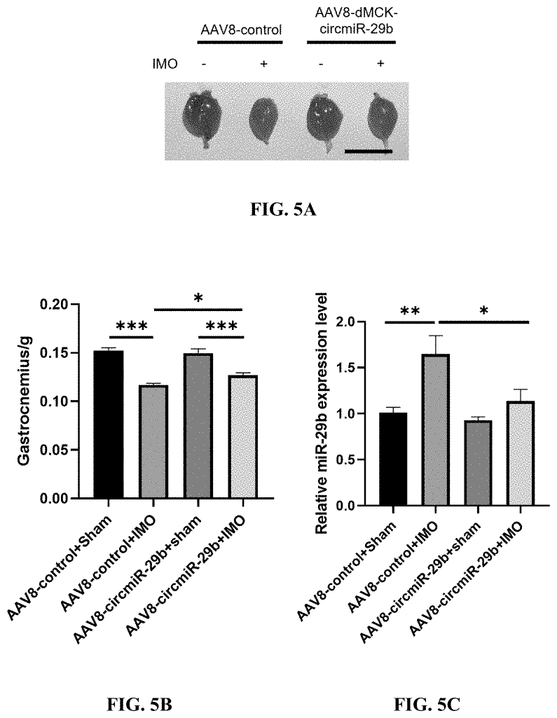

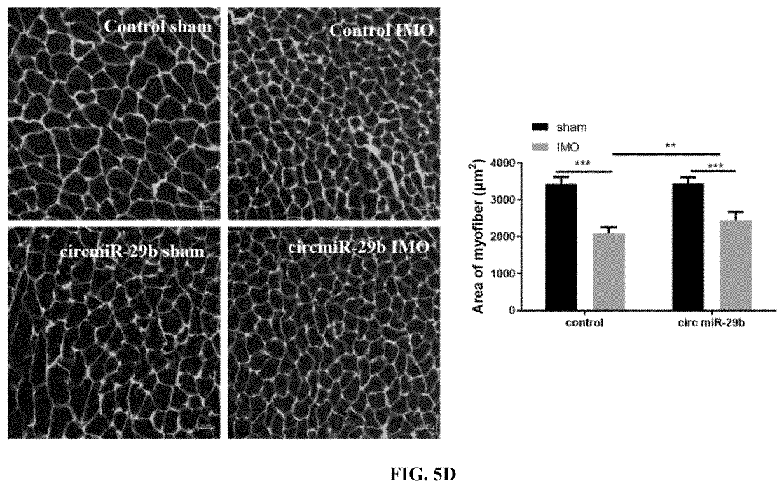

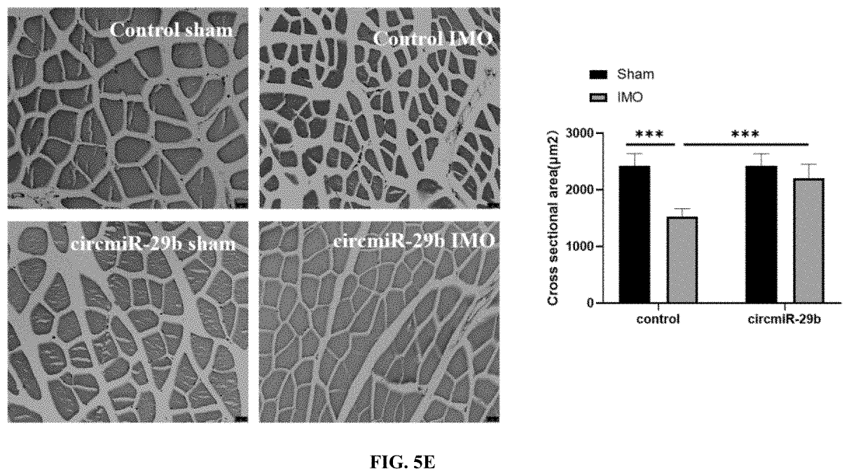

The results are shown in FIG. 5. The mouse gastrocnemius samples are significantly atrophied in the IMO model, and circmiR-29b can significantly protect the muscle from weight loss (FIG. SA and FIG. 5B). Fluorescence quantitative PCR results show that after the IMO model mice are injected with AAV8-circmiR-29b, the miR-29b expression is significantly reduced (FIG. SC). WGA and HE staining show that after the IMO model mice are injected with AAV8-circmiR-29b, the diameter of myofiber is obviously improved (FIG. 5D and FIG. SE). It can be seen that the circmiR-29b delivered by AAV enable protection from the muscle atrophy induced by the IMO model.

### EXAMPLE 7

1. Construction of Angil (Angil sustained release pump) induced muscle atrophy model.

Wild-type male mice of 8-10 weeks old were injected intraperitoneally with 1% sodium pentobarbital at a dose of 10 μL/g for anesthesia. After anesthesia, the mice were fixed on a thermostat blanket with their abdomen facing down using a medical tape, and the hairs on the back neck were removed with depilatory cream The depilated part was sterilized with 75% ethanol, and then an incision was cut 3 mm long on the back with microscissors. The Alzet sustained release pump filled with 200 μL of 2 mg/mL Angil solution was inserted subcutaneously through the gap on the back. The pump was Model 2001 (ALZET) with Angil release efficiency of 1.46 mg/kg/day. The sustained release pump in the control group was filled with PBS. Subsequently, the epidermis was sutured, and the mice were kept warm on the thermostat blanket before resuscitation. Then the mice were transferred to cages and raised for 7 d before they were sacrificed. The mice were measured for body weight, tibia length, and gastrocnemius weight, and then the gastrocnemius samples were kept for subsequent detection. 1 week after the model was constructed, the mice were dissected, and the gastrocnemius was weighed. The weight of the gastrocnemius with successfully constructed model decreased significantly.

2. The mouse gastrocnemius was injected with AAVS-circmiR-29 virus. A gene fragment capable of synthesizing circmiR-29b was integrated into an AAV8 vector and packaged into sufficient AAV8 viruses with the help of AAV8 capsid plasmid and Helper plasmid. After separation and purification, the AAV viral titer was determined. The virus was injected into the gastrocnemius in situ at a dose of 1×1012 VG/mouse. 3 weeks later, construction of muscle atrophy model was carried out.

A specific method was as follows:

3 weeks before the Angil model was constructed, the mice were treated by intramuscular injection with a disposable 1 mL sterile syringe. The test included 4 groups, and was carried out with a specific method as follows:

for group 1, the control virus+PBS group, the mouse gastrocnemius was injected with the control virus at a dose of 50 μL/mouse, and a pump was embedded 3 weeks later;

for group 2, the control virus+Angil group, the mouse gastrocnemius was injected with the control virus at a dose of 50 μL/mouse, and a pump was embedded 3 weeks later;

for group 3, the AAV8-circmiR-29b virus+PBS group, the mouse gastrocnemius injected with the AAV8-circmiR-29b virus, and a pump was embedded 3 weeks later;

for group 4, the AAV8-circmiR-29b virus+Angil group, the mouse gastrocnemius was injected with the AAV8-circmiR-29b virus, and a pump was embedded 3 weeks later.

1 week later, the test was over, and the mice were sacrificed. The gastrocnemius was obtained through dissection and weighed with an analytical balance. Then the muscle samples were subjected to WGA staining and HE staining to calculate the change in the cross-sectional area of myofiber. Meanwhile, tissue samples were taken. Total RNA of gastrocnemius tissue was extracted, and the change of miR-29b expression was detected by fluorescence quantitative PCR.

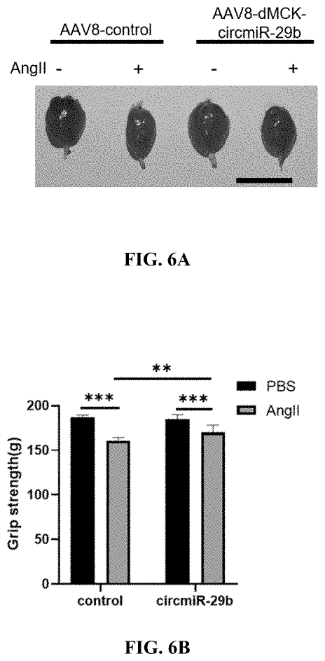

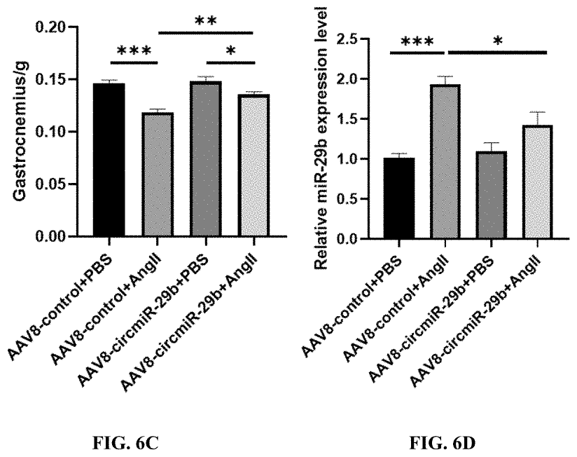

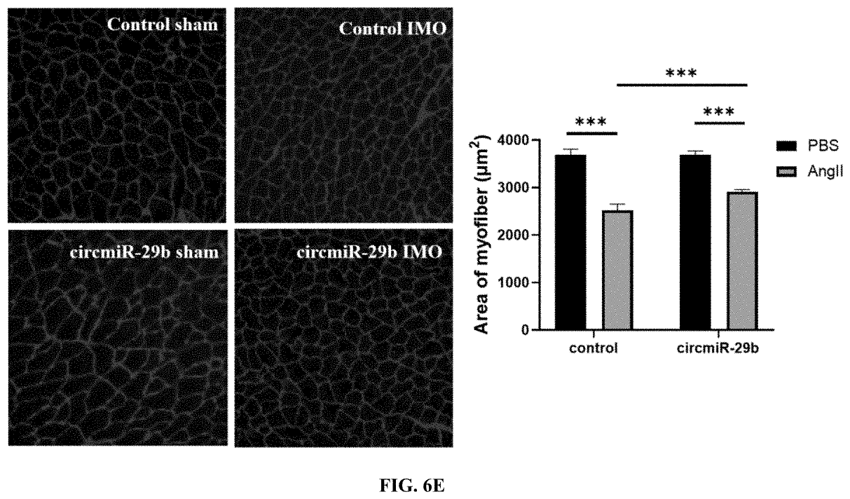

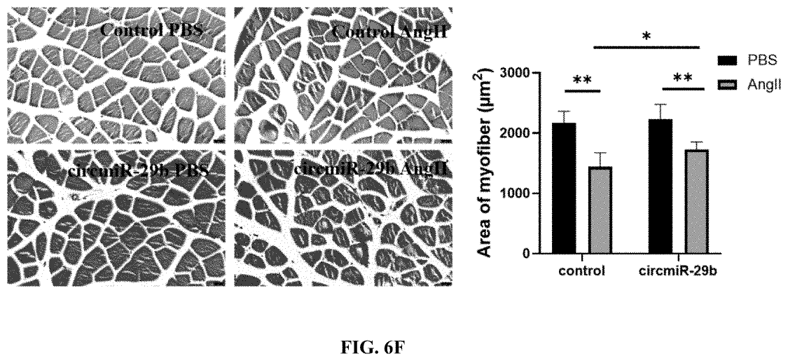

The results are shown in FIG. 6. In the Angil model, circmiR-29b can result in significant recovery of mouse grip strength and protect the muscle from weight loss (FIG. 6A-FIG. 6C). Fluorescence quantitative PCR results show that after the Angil model mice are injected with AAV8-circmiR-29b, the miR-29b expression is significantly reduced (FIG. 6D). WGA and HE staining shows that after the IMO model mice are injected with AAV8-circmiR-29b, the diameter of myofiber is obviously improved (FIG. 6E and FIG. 6F). It can be seen that the delivered circmiR-29b enable protection from the muscle atrophy induced by the Angil sustained release (Angil) model.

### EXAMPLE 8

1. Construction of SWI induced muscle atrophy model.

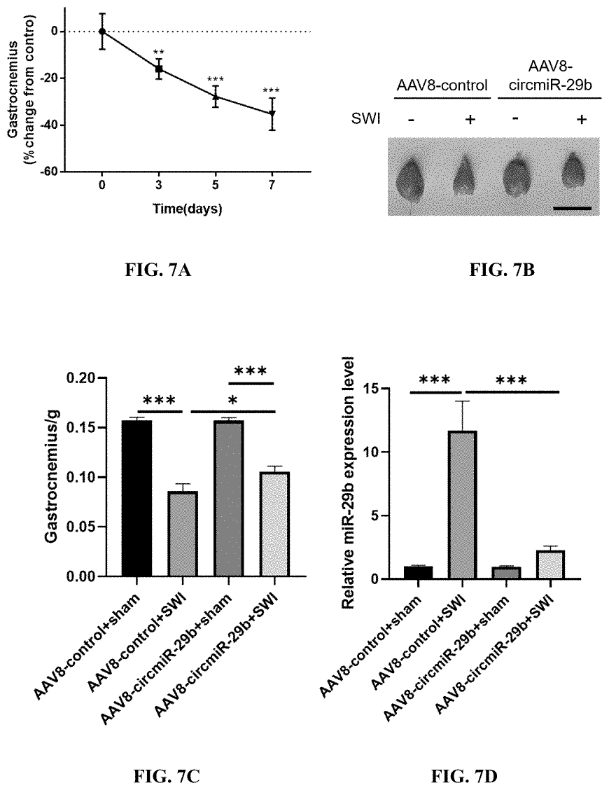

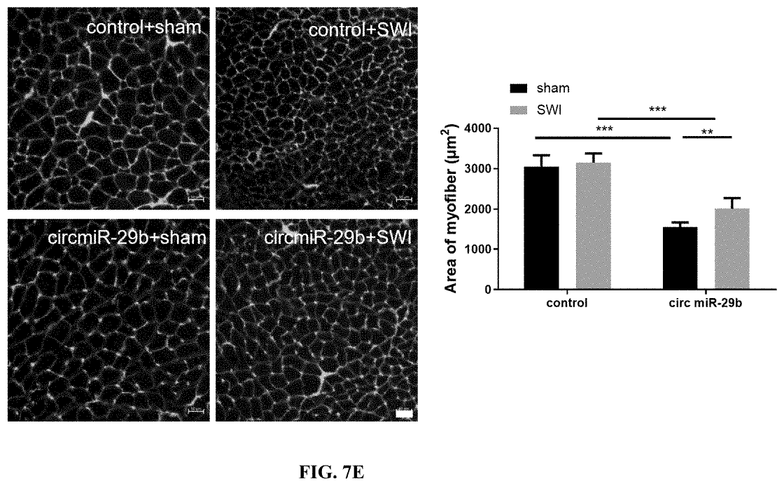

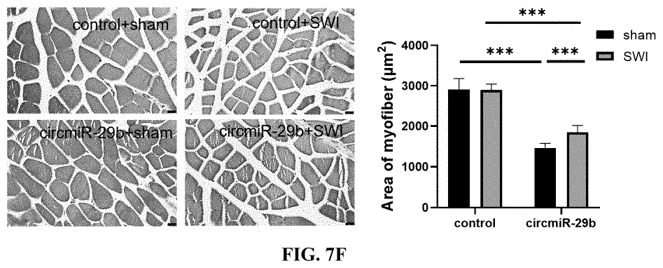

Wild-type male mice of 8-10 weeks old were injected intraperitoneally with 1% sodium pentobarbital at a dose of 10 μL/g for anesthesia. After anesthesia, the mice were fixed on a thermostat blanket with their abdomen facing down using a medical tape. The mouse hind limbs were wound with a medical tape not too tightly, avoiding affecting normal blood flow. Subsequently, the hind limbs of the mice were spirally wound and immobilized with a 2 mm thick wire, while the mice in the control group moved freely. The mice were sacrificed at day 3, 5 or 7 after immobilization, and the gastrocnemius was obtained by dissection and weighed. The results showed that the gastrocnemius was significantly atrophied on day 3 after immobilization, and the muscle atrophy was gradually intensified with the extension of the immobilization time (FIG. 7A). This indicated that the mouse muscle atrophy model was successfully constructed.

2. On day 4 after the construction of the SWI model when muscle atrophy had occurred, the mouse gastrocnemius was injected with AAVS-circmiR-29 virus. A gene fragment capable of synthesizing circmiR-29b was integrated into an AAV8 vector and packaged into sufficient AAV8 viruses with the help of AAV8 capsid plasmid and Helper plasmid. After separation and purification, the AAV viral titer was determined. The virus was injected into the gastrocnemius in situ at a dose of 1×1012 VG/mouse. 4 weeks later, the mice were sacrificed and measured for body weight, tibia length, and gastrocnemius weight. Then the gastrocnemius samples were kept for subsequent detection.

A specific method was as follows:

On day 4 after the construction of the SWI model, the mice were treated by intramuscular injection with a disposable 1 mL sterile syringe. The test included 4 groups, and was carried out with a specific method as follows:

for group 1, the control virus+control group, the mouse gastrocnemius was injected with the control virus at a dose of 50 μL/mouse;

for group 2, the control virus+SWI group, the mouse gastrocnemius was injected with the control virus at a dose of 50 μL/mouse;

for group 3, the AAV8-circmiR-29b virus+control group, the mouse gastrocnemius was injected with the AAV8-circmiR-29b virus;

for group 4, the AAV8-circmiR-29b virus+SWI group, the mouse gastrocnemius was injected with the AAV8-circmiR-29b virus.

4 week later, the test was over, and the mice were sacrificed. The gastrocnemius was obtained through dissection and weighed with an analytical balance. Then the muscle samples were subjected to WGA staining and HE staining to calculate the change in the cross-sectional area of myofiber. Meanwhile, tissue samples were taken. Total RNA of gastrocnemius tissue was extracted, and the change of miR-29b expression was detected by fluorescence quantitative PCR.

The results are shown in FIG. 7. In the SWI model, circmiR-29b can protect the muscle from weight loss (FIG. 7B -FIG. 7C). Fluorescence quantitative PCR results show that after the SWI model mice are injected with AAV8-circmiR-29b, the miR-29b expression is significantly reduced (FIG. 7D). WGA and HE staining shows that after the IMO model mice are injected with AAV8-circmiR-29b, the diameter of myofiber is obviously improved (FIG. 7E and FIG. 7F). It can be seen that the delivered circmiR-29b enable protection from the muscle atrophy induced by the SWI model.

The above descriptions are merely preferred embodiments of the present disclosure. It should be noted that a person of ordinary skill in the art may further make several improvements and modifications without departing from the principle of the present disclosure, but such improvements and modifications should also be regarded as falling within the protection scope of the present disclosure.

1. A circular RNA circmiR-29b, wherein the circular RNA circmiR-29b comprises an effective sequence and a random sequence, 6-13 repetitions of the effective sequence are connected in series, the random sequence is inserted between the effective sequences; and wherein the nucleotide sequence of the effective sequence is shown in SEQ ID NO: 1.

2. The circular RNA circmiR-29b according to claim 1, wherein 10-12 repetitions of the effective sequence are connected in series.

3. The circular RNA circmiR-29b according to claim 1, wherein the random sequence comprises 12-24 nucleotides (nt).

4. The circular RNA circmiR-29b according to claim 3, wherein the nucleotide sequence of the random sequence is shown in SEQ ID NO: 2.

5. The circular RNA circmiR-29b according to claim 1, wherein the nucleotide sequence of the circular RNA circmiR-29b is shown in SEQ ID NO: 3.

6. A recombinant adeno-associated virus (AAV) vector, comprising the circular RNA circmiR-29b of claim 1.

7. The recombinant AAV vector according to claim 6, wherein the AAV vector comprises an AAV8 vector.

8. The recombinant AAV vector according to claim 6, wherein the AAV vector further comprises a 5′ guided loop-forming sequence and a 3′ guided loop-forming sequence;
wherein the nucleotide sequence of the 5′ guided loop-forming sequence is shown in SEQ ID NO: 4;
wherein the nucleotide sequence of the 3′ guided loop-forming sequence is shown in SEQ ID NO: 5;
and wherein the circular RNA circmiR-29b is inserted between the 5′ guided loop-forming sequence and the 3′ guided loop-forming sequence.

9. A delivery system for a circular RNA circmiR-29b according to claim 1, wherein the delivery system is an AAV containing the circular RNA circmiR-29b of claim 1.

10. Use of the circular RNA circmiR-29b according to claim 1, in preparation of a medicine for preventing and/or treating muscle atrophy.

11. A delivery system for a circular RNA circmiR-29b according to claim 1, wherein the delivery system is obtained by packaging the recombinant AAV vector of claim 6.

12. Use of the recombinant AAV vector according to claim 6 in preparation of a medicine for preventing and/or treating muscle atrophy.

13. Use of the delivery system for a circular RNA circmiR-29b according to claim 9 in preparation of a medicine for preventing and/or treating muscle atrophy.
